(19) United States
(12) Patent Application Publication
(10) Pub. No.: US 2022/0163435 A1
(43) Pub. Date: May 26, 2022

(54) UNIVERSAL FOLDING TEST DEVICE FOR FOLDABLE DISPLAY

(71) Applicant: SP Technology Co., Ltd., Anyang-si (KR)

(72) Inventor: Yong Jin Park, Uiwang-si (KR)

(73) Assignee: SP Technology Co., Ltd.

(21) Appl. No.: 17/114,810

(22) Filed: Dec. 8, 2020

(30) Foreign Application Priority Data
Nov. 24, 2020 (KR) 10-2020-0159388

(51) Int. Cl. G01N 3/20

(52) U.S. Cl. CPC G01N 3/20; G01N 2203/0037; G01N 2203/0023

(57) ABSTRACT

A universal folding test device for a foldable display is proposed. The universal folding test device includes: a support plate having a flat-plate shape, being arranged horizontally with a Z axis as a normal line in a space formed by three coordinate axes of X, Y, and Z that are orthogonal to each other, and having opposite sides thereof formed parallel to the Y axis; and a rotary plate being arranged symmetrically on each of the opposite sides of the support plate, being formed as a flat plate having a side thereof adjacent to the support plate and parallel to the Y axis, having a Y-axis rotation actuator formed thereon where the side adjacent to the support plate becomes a rotary shaft, and having a X and Z-axis transfer actuator coupled thereto, wherein a foldable display substrate is attached to the support plate and the rotary plate.

### REFERENCE TO RELATED APPLICATIONS

This application claims the priority benefit of Korean Patent Application No. 10-2020-0159388 filed on Nov. 24, 2020 the entire contents of which are incorporated herein by reference.

### FIELD OF THE INVENTION

The present disclosure relates to a universal folding test device for a foldable display and, more particularly, to a folding test device capable of simultaneously performing a test for two-stage folding of a foldable display that folds twice in two stages in an environmental test chamber and capable of freely selecting an in-folding or out-folding test while being able to freely determine an in-folding radius or out-folding radius under a condition of the two-stage folding.

### BACKGROUND OF THE INVENTION

A foldable display is a display manufactured using a thin and flexible substrate that is able to be curved, bent, or rolled.

Accordingly, unlike conventional flat panel displays, in a foldable display, a folding test to examine folding performance is performed during manufacturing.

Meanwhile, foldable displays are classified into two types in terms of folding method: an in-folding type in which display surfaces of a substrate are folded to face with each other; and an out-folding type in which rear surfaces of a substrate are folded to face with each other. Recently, a hybrid type in which the in-folding and out-folding types are simultaneously applied to a single display at the same time has been developed.

Accordingly, when manufacturing a foldable display substrate, depending on a design type of the display, a folding test for the foldable substrate is performed by an in-folding test in which the display surfaces of the foldable substrate are folded to face with each other, an out-folding test in which the rear surfaces of the boards are folded to face with each other, or a hybrid folding test in a combined form of the in-folding and out-folding tests.

However, conventionally, when the in-folding test and the out-folding test are performed in a single folding test device, there is a problem in that it is difficult to conduct the tests due to a difference between thicknesses of substrates or a difference between physical structures (i.e., hinge structures) of substrates.

For a folding test of a foldable display, technologies disclosed in Korean Patent No. 10-1901390, Korean Patent No. 10-1843874, Korean Patent No. 10-1489667, etc. have been previously developed. In particular, a multi folding tester disclosed in Korean Patent No. 10-2088613 “MULTI FOLDING TESTER” was developed as a means for testing both the in-folding test and out-folding test with a single folding test device.

However, all such methods of the related art are developed as solutions to a display in which one folding is performed on the display, and thus there is a problem in that the solutions are unable to be used in a device of simultaneous folding test for a foldable display, wherein the foldable display is designed such that folding of two parts of a display is performed, but designed for a case where both parts are all in-folded or both parts are all out-folded, or for a case where one part is in-folded and the other part is out-folded.

### Documents of Related Art

(Patent Document 1) Korean Patent No. 10-1901390
(Patent Document 2) Korean Patent No. 10-1843874
(Patent Document 3) Korean Patent No. 10-1489667
(Patent Document 4) Korean Patent No. 10-2088613

### SUMMARY OF THE INVENTION

An embodiment of the present disclosure is to solve the above problems, and an objective of the present disclosure is to provide a universal folding test device for a foldable display, wherein, by using a single folding test device, two-part folding tests are simultaneously performed on a single foldable display in which two parts are folded, and a universal folding test may be performed for all foldable displays which are designed for a case where both parts are all in-folded or both parts are all out-folded, or for a case where one part is in-folded and the other part is out-folded.

In order to achieve the above objective, the embodiment of the present disclosure uses a universal folding test device for a foldable display as a technical subject matter, the universal folding test device including: a support plate having a flat-plate shape, being arranged horizontally with a Z axis as a normal line in a space formed by three coordinate axes of X, Y, and Z that are orthogonal to each other, and having opposite sides thereof formed parallel to the Y axis; and a rotary plate being arranged symmetrically on each of the opposite sides of the support plate, being formed as a flat plate having a side thereof adjacent to the support plate and parallel to the Y axis, having a Y-axis rotation actuator formed on the side adjacent to the support plate, and having a X and Z-axis transfer actuator coupled thereto, wherein a foldable display substrate is attached to the support plate and the rotary plate, a folding radius is adjusted by the X and Z-axis transfer actuators, and an in-folding or out-folding test is performed by controlling the Y-axis rotation actuator.

In addition, it is preferable to realize the universal folding test device for the foldable display, wherein the Y-axis rotation actuator may include: a yoke having a first side thereof coupled to the rotary shaft of the rotary plate as a driven shaft and having a second side thereof connected to a rotation shaft to be coupled as a driving shaft; and a reduction gear and a rotation motor, which are connected to the rotation shaft.

In addition, it is preferable to realize the universal folding test device for the foldable display, wherein the X and Z-axis transfer actuator may include: a yoke mount having an upper side thereof coupled to the yoke and having a side surface thereof on which a Z-axis slider is formed; a X and Z-axis frame provided with a vertical plate on which a Z-axis guide rail is formed, so as to be coupled to the Z-axis slider, and provided with a horizontal plate being coupled at a right angle to a lower side of the vertical plate and having a lower side thereof on which an X-axis slider is formed; and a main mount having an upper surface thereof on which an X-axis guide rail is formed, so as to be coupled to the X-axis slider.

In addition, it is preferable to realize the universal folding test device for the foldable display, wherein the rotary plate, the yoke, and the support plate may be formed inside an environmental chamber, the Y-axis rotation actuator may be provided with the rotation shaft extending to an outer side of the environmental chamber so that the reduction gear and the rotation motor may be formed at the outer side of the environmental chamber, and the X and Z-axis transfer actuator may be provided with a yoke support rod extending to the outer side of the environmental chamber and coupled to a lower side of the yoke so that the yoke mount may be formed at the outer side of the environmental chamber, whereby a folding test with the rotary plate and the support plate may be able to be performed under environmental conditions.

In addition, it is preferable to realize the universal folding test device for the foldable display, wherein the yoke mount may further include a rotation shaft support that extends vertically to the rotation shaft at the outer side of the environmental chamber, so as to support the rotation shaft.

The embodiment of the present disclosure uses the universal folding test device for the foldable display as another technical subject matter, wherein two universal folding test devices for the foldable display may be symmetrically formed to face with each other, and only the rotary plate, the yoke, and the support plate may be formed inside the environmental chamber.

According to the embodiment of the present disclosure described above, there is an effect in that a universal folding test device for a foldable display is provided, wherein, by using a single folding test device, two-part folding tests are simultaneously performed on a single foldable display in which two parts are folded, and a universal folding test may be performed for all foldable displays which are designed for a case where both parts are all in-folded or both parts are all out-folded, or for a case where one part is in-folded and the other part is out-folded.

### BRIEF DESCRIPTION OF THE DRAWINGS

FIG. 1 is a structural view of an embodiment of the present disclosure.

FIG. 2 is an overall perspective view showing the exemplary embodiment of the present disclosure.

FIG. 3 is a perspective side view showing the exemplary embodiment of the present disclosure.

FIG. 4 is a front side view showing the exemplary embodiment of the present disclosure.

FIG. 5 is a perspective view showing an environmental chamber coupled to the exemplary embodiment of the present disclosure.

### DETAILED DESCRIPTION OF THE INVENTION

An embodiment of the present disclosure will be described below with reference to the drawings, and in the following description of the embodiment of the present disclosure, detailed descriptions of known functions and components incorporated herein will be omitted when it may make the subject matter of the embodiment of the present disclosure unclear.

In addition, since terms to be described later are terms defined in consideration of functions in the embodiment of the present disclosure, which may vary according to the intentions or practices of users or operators, the definition of the terms should be made based on the contents throughout the present specification describing the embodiment of the present disclosure.

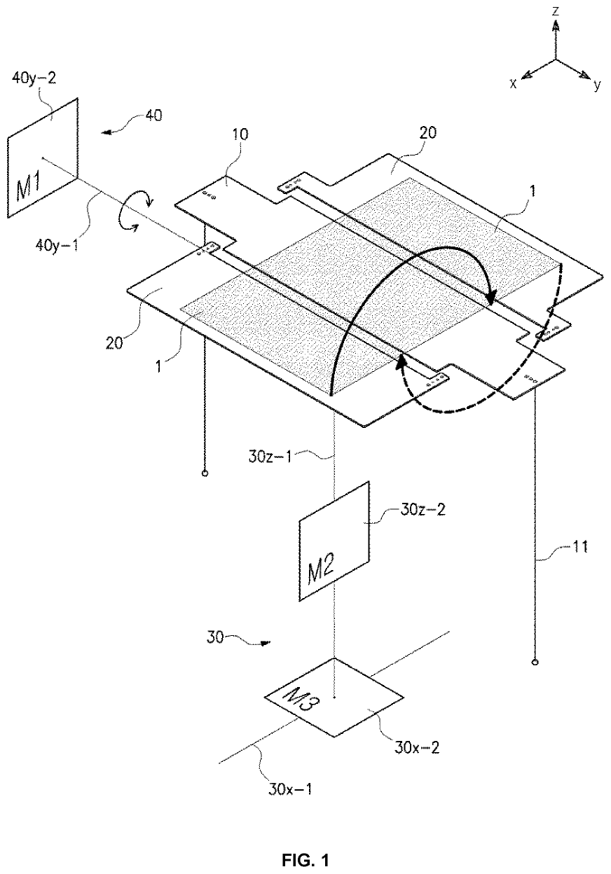

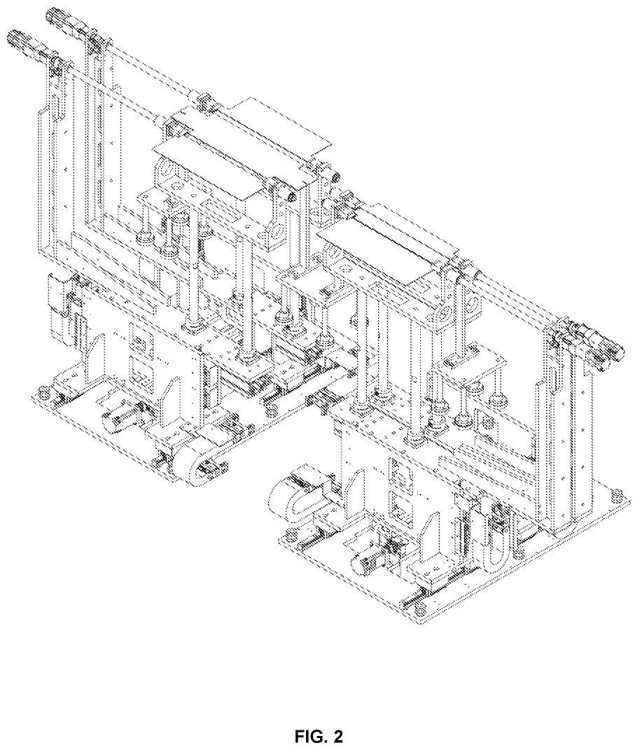

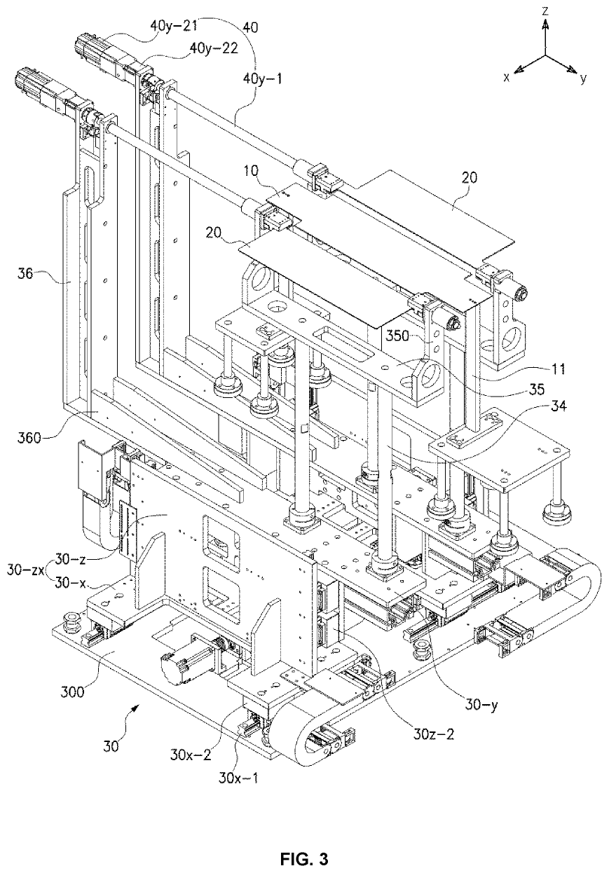

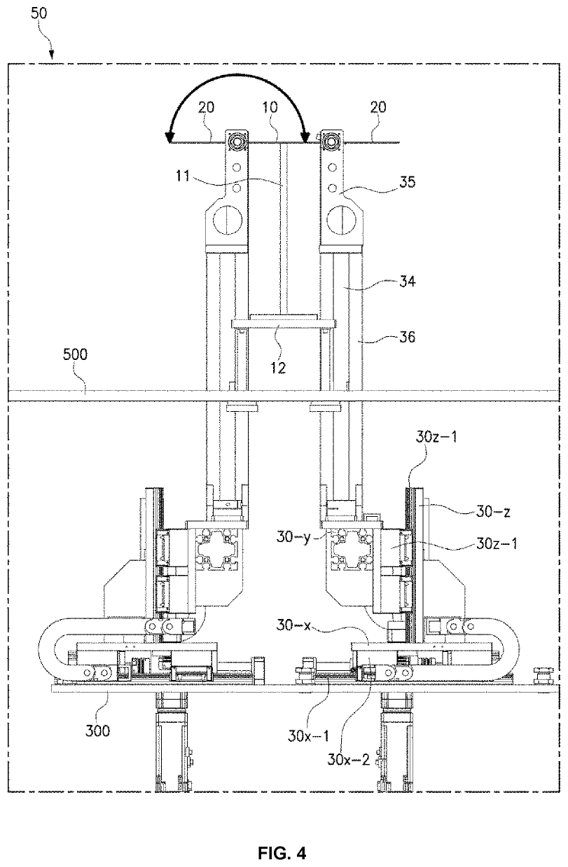

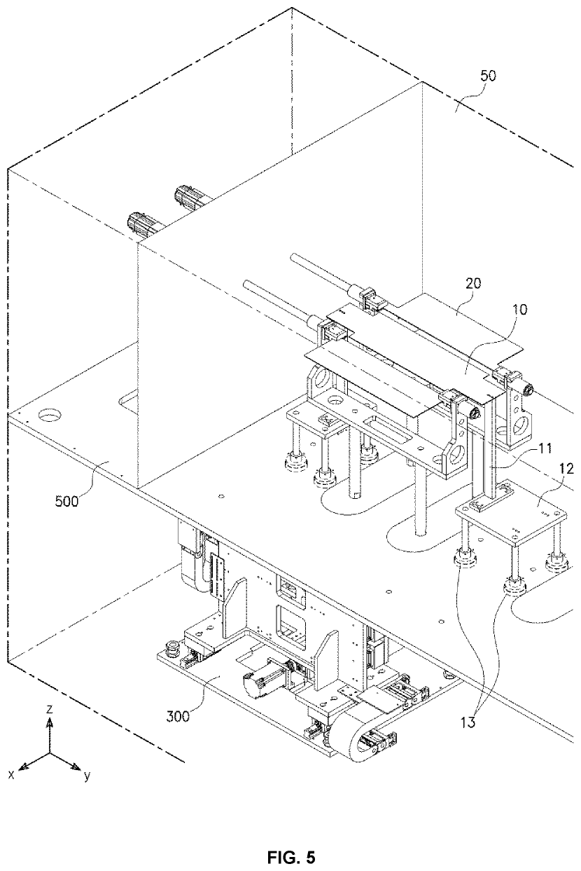

Hereinafter, FIG. 1 is a structural view of an embodiment of the present disclosure, FIG. 2 is an overall perspective view showing the exemplary embodiment of the present disclosure, FIG. 3 is a perspective side view showing the exemplary embodiment of the present disclosure, FIG. 4 is a front side view showing the exemplary embodiment of the present disclosure, and FIG. 5 is a perspective view showing an environmental chamber coupled to the exemplary embodiment of the present disclosure.

The present disclosure relates to a test device for a foldable display in which two parts are folded in a single display.

Accordingly, in the embodiment of the present disclosure, a folding plate that is folded by attaching a display is divided into three parts, wherein a support plate 10 is positioned in a center of the folding plate, and rotary plates 20 folding the display 1 are symmetrically positioned on opposite sides of the support plate 10.

As shown in FIG. 1, in a space formed by three coordinate axes of X, Y, and Z that are orthogonal to each other, the support plate 10 is horizontally arranged with the Z axis as a normal, is formed in a flat plate having opposite sides thereof each parallel to the Y axis, and is fixed by each support 11 extending vertically at a lower side of the support plate 10.

The rotary plate 20 is symmetrically arranged on each of the opposite sides of the support plate 10 and is formed of a flat plate having a side adjacent to the support plate 10, the side being parallel to the Y axis.

According to this configuration, each of the rotary plates 20 is symmetrically formed on each of the opposite sides of the support plate 10, and every side where the support plate 10 and each of the rotary plates 20 are adjacent to each other is formed parallel to the Y axis.

In the embodiment of the present disclosure, each of the rotary plates 20 is configured to include: a Y-axis rotation actuator 40 for rotating sides adjacent to the support plate 10 in the Y-axis direction (i.e., a direction of rotating around the Y-axis); and an X and Z-axis transfer actuator 30 for transferring all rotary plates 20 in the X and Z-axis directions.

As described above, since the rotary plates 20 are symmetrically formed centering around the support plate 10, the Y-axis rotation actuator 40 and the X and Z-axis transfer actuator 30 are also symmetrically formed.

According to such a configuration, after attaching a foldable display to the support plate 10 and each rotary plate 20 in the X-axis direction, when rotating the rotary plate 20 on each of the opposite sides of the support plate 10, a folding test is performed for the foldable display 1.

Accordingly, as shown in FIG. 1, the folding test is performed on the opposite sides of the support plate 10 at the same time, and the Y-axis rotation actuator 40 is 360 degree rotatable so that each folding test part is selectively operated for an in-folding test or out-folding test depending on a direction of controlled axis rotation.

Meanwhile, during a folding test, the folding part has a folding radius for in-folding and a folding radius for out-folding, which are different from each other, and when two folding parts are all in-folded or are all out-folded, the folding radii become different.

In the embodiment of the present disclosure, the folding radii of the two parts, which are different from each other depending on such a folding type, are independently formed by the X and Z-axis transfer actuators 30.

That is, when the rotary plate 20 is moved with respect to the support plate 10 by the X and Z-axis transfer actuator 30, every side where the support plate 10 and each of the rotary plates 20 are adjacent to each other is moved in the X and Z-axis directions, and accordingly the folding radii of the rotary plates 20 and the support plate 10 are changed.

Therefore, in the embodiment of the present disclosure, according to the fact that whether two folding parts are in-folded or out-folded, or whether the two folding parts are folded first in the case where the two folding parts are all in-folded or are all out-folded, the folding radii are set by the X and Z-axis transfer actuator 30, and the folding test is performed by driving the Y-axis rotation actuator 40.

Accordingly, the embodiment of the present disclosure is characterized in that each folding radius is set for a single display 1 that is folded in two parts, and then a folding test is performed.

In the embodiment of the present disclosure shown in FIG. 3, the Y-axis rotation actuator 40 is configured to include: a yoke 35, a rotation shaft 40y-1, a reduction gear 40y-22, and a rotation motor 40y-21.

In the yoke 35, a rotary shaft of the rotary plate 20 is coupled to a first yoke arm 350 as a driven shaft, and a rotation shaft 40y-1 is connected to a second yoke arm 350 to be coupled as a driving shaft.

When the rotary plate 20 is rotated like a hinge with respect to the support plate 10, the rotary shaft of the rotary plate 20 refers to an axis that operates like a hinge axis. In the yoke 35, the first side thereof is coupled as a driven shaft and the second side thereof is coupled as a driving shaft.

The driving shaft is implemented by coupling to a rotation shaft 40y-1 driven by connecting to the rotary plate 20. The reduction gear 40y-22 and the rotation motor 40y-21 are coupled to the rotation shaft 40y-1 to provide driving force.

Accordingly, in the embodiment of the present disclosure, when the rotation motor 40y-21 is driven and controlled, the rotary plate 20 is rotated as the rotation shaft 40y-1 is rotated.

Since the objective of the embodiment of the present disclosure is to provide a folding test of the display 1, as shown in FIG. 1, the driving of the rotation motor 40y-21, the reduction gear 40y-22, and the rotation shaft 40y-1 allows the display 1 to be repeatedly folded.

In addition, as shown in FIG. 1, depending on whether the folding test part is in-folded or out-folded, the reciprocation of each predetermined section is performed in a way of reciprocating the display in contact with an upper surface of the support plate 10 or reciprocating the display in contact with a bottom surface of the support plate 10.

As shown in FIG. 3, when selecting such an in-folding or out-folding section, the yoke 35 to which the rotary plate 20 is coupled is operated as a support that does not interfere with the rotation.

Hereinafter, together with FIGS. 3 and 4, an exemplary embodiment of the X and Z-axis transfer actuator 30 of the present disclosure will be described in detail.

As shown in FIGS. 3 and 4, the X and Z-axis transfer actuator 30 is configured on a yoke mount 30-y and an X and Z-axis frame 30-zx.

The yoke mount 30-y is a support for mounting the yoke 35, and in the exemplary embodiment of the present disclosure, the yoke mount 30-y is composed of a hexahedral rod arranged in the Y-axis direction, wherein the yoke 35 is coupled to an upper side of the yoke mount 30-y and a Z-axis slider 30z-2 is coupled to a side surface of the yoke mount 30-y.

The X and Z-axis frame 30-zx is composed of two vertically orthogonal plate frames, and normal lines of each plate are respectively formed parallel to the X axis and the Z axis.

At this time, the plate whose normal line is parallel to the X axis is a vertical plate 30-z, which becomes a Z-axis frame, and the plate whose normal line is parallel to the Z axis is a horizontal plate 30-x, which becomes an X-axis frame, whereby an assembled body of the two frames is defined as the X and Z-axis frame 30-zx.

As shown in FIG. 4, a Z-axis guide rail 30z-1 is formed on the vertical plate 30-z, so as to be coupled to the Z-axis slider 30z-2.

The Z-axis guide rail 30z-1 is a rail installed parallel to the Z-axis on the vertical plate 30-z, and a Z-axis slider 30z-2 is coupled to the Z-axis guide rail 30z-1, whereby the yoke mount 30-y is driven upward and downward in the Z-axis direction.

The horizontal plate 30-x is coupled at a right angle to a lower side of the vertical plate 30-z, and is coupled to an X-axis slider 30x-2 in parallel with the X-axis at a lower side of the horizontal plate 30-x.

A main mount 300 of the present disclosure is a basic flat plate on which the devices of the present disclosure are built, and an X-axis guide rail 30x-1 having an upper side thereof to which the X-axis slider 30x-2 is coupled is formed in the X-axis direction.

Accordingly, by driving of the X-axis slider 30x-2, the vertical plate 30-z, the yoke mount 30-y, the yoke 35, and the rotary plate 20, which are mounted on the horizontal plate 30-x as well as the horizontal plate 30-x are moved in the X-axis.

When driving the X and Z-axis transfer actuator 30 of the present disclosure configured as described above, each rotary plate 20 may be moved in the X and Z-axis, and this means that a position of the rotary shaft of the rotary plate 20 may be changed.

As described above, since the position of the rotary shaft determines a folding radius of the display 1 to be tested, the X and Z-axis transfer actuator 30 is driven according to the desired in-folding method, out-folding method, and folding levels, thereby adjusting the folding radius.

In the description of the present disclosure, since the driving part for driving the X-axis slider 30x-2 and the Z-axis slider 30z-2, which are components of the X and Z-axis transfer actuators 30, is a ready-made product and corresponds to a known technology, a detailed description will be omitted.

Meanwhile, in the embodiment of the present disclosure, since a folding radius is adjusted by the driving of the X and Z-axis transfer actuator 30, it is preferable to include a controller configured to automatically transfer the rotary shaft of the rotary plate 20 by outputting a control command to the X-axis slider 30x-2 and the Z-axis slider 30z-2 when a folding radius is input.

In the same manner as a general display 1, the folding test of a foldable display also requires a test under environmental conditions in consideration of the climate change.

In view of this requirement, the embodiment of the present disclosure allows the folding test device for a foldable display to be put into an environmental chamber 50 for testing.

However, since the part exposed to the test environment is the foldable display substrate which is the part that needs to be tested, and since device failure is caused or device design costs are increased when other mechanical components are exposed to the environmental test conditions, only the folding part of the foldable display that needs to be tested is built into the environmental chamber 50 in the embodiment of the present disclosure, and other mechanical parts are installed outside the environmental chamber 50, thereby ensuring reliability of device operation.

To this end, in the embodiment of the present disclosure, the rotary plate 20, the yoke 35, and the support plate 10 are configured to be formed in the environmental chamber 50 which has a lattice shape, and the Y-axis rotation actuator 40 allows the rotation shaft 40y-1 to extend long toward an outer side of the environmental chamber 50, and then couples the rotation shaft 40y-1 to both the reduction gear 40y-22 and the rotation motor 40y-21 at the outer side of the environmental chamber 50, so as to rotate the reduction gear 40y-22 and the rotation motor 40y-21.

In addition, in the X and Z-axis transfer actuator 30, a yoke support rod 34 extending to the outer side of the environmental chamber 50 is coupled to a lower side of the yoke 35, so that the yoke mount 30-y is formed at the outer side of the environmental chamber 50.

Accordingly, the X and Z-axis transfer actuator 30 coupled to the yoke mount 30-y is also formed at the outer side of the environmental chamber 50 to ensure operational reliability at room temperature.

For this reason, as for the support plate 10, since the support plate 10 is only fixed by the support 11 extendedly formed vertically to the lower side of the support plate 10 and is not couple to the transfer actuator, etc., it is sufficient to fix the support 11 to an inner floor of the environmental chamber 50.

Meanwhile, as for the rotation shaft 40y-1, since the rotation shaft 40y-1 extends long toward the outer side of the environmental chamber 50, there is a fear that the rotation shaft 40y-1 may be bent by its own weight, and thus it is necessary to support and reinforce an end of the rotation shaft 40y-1.

To this end, in the embodiment of the present disclosure, one side of the yoke mount 30-y installed at the outer side of the environmental chamber 50 extends vertically in the direction of the rotation shaft 40y-1, so as to be formed as a rotation shaft support 36 which supports the rotation shaft 40y-1.

In the exemplary embodiment of the present disclosure, the rotary shaft 40y-1 and the rotary shaft support 36 are coupled to each other via a bearing, and a reinforcing bar 360 supporting a vertical coupling is coupled to both positions between the rotation shaft support 36 and the yoke mount 30-y.

In the embodiment of the present disclosure, since the foldable display 1 is tested inside the environmental chamber as described above, an efficient way of using the environmental chamber 50 is required.

To this end, in the embodiment of the present disclosure, two universal folding test devices for a foldable display are formed to face with each other symmetrically, and only the rotary plate 20, the yoke 35, and the support plate 10 are provided inside the environmental chamber 50.

Accordingly, there is an advantage in that two display tests may be performed using the limited space inside the environmental chamber 50.

As such, the drawings shown above for the description of the present disclosure are one exemplary embodiment in which the present disclosure is realized, and as shown in the drawings, it may be seen that various types of combinations are possible in order to realize the subject matter of the present disclosure.

Therefore, the embodiment of the present disclosure is not limited to the above-described exemplary embodiments, and as claimed in the following claims, it will be said that there is a technical spirit of the present disclosure to the extent in which various changes can be implemented by anyone of those skilled in the art to which the present disclosure belongs without departing from the gist of the present disclosure.

What is claimed is:

1. A universal folding test device for a foldable display, the universal folding test device comprising:
a support plate having a flat-plate shape, being arranged horizontally with a Z axis as a normal line in a space formed by three coordinate axes of X, Y, and that are orthogonal to each other, and having opposite sides thereof formed parallel to the Y axis; and
a rotary plate being arranged symmetrically on each of the opposite sides of the support plate, being formed as a flat plate having a side thereof adjacent to the support plate and parallel to the Y axis, having a Y-axis rotation actuator formed thereon where the side adjacent to the support plate becomes a rotary shaft, and having a X and Z-axis transfer actuator coupled thereto,
wherein a foldable display substrate is attached to the support plate and the rotary plate, a folding radius is adjusted by the X and Z-axis transfer actuators, and an in-folding or out-folding test is performed by controlling the Y-axis rotation actuator.

2. The universal folding test device of claim 1, wherein the Y-axis rotation actuator comprises:
a yoke having a first side thereof coupled to the rotary shaft of the rotary plate as a driven shaft and having a second side thereof connected to a rotation shaft to be coupled as a driving shaft; and
a reduction gear and a rotation motor, which are connected to the rotation shaft.

3. The universal folding test device of claim 2, wherein the X and Z-axis transfer actuator comprises:
a yoke mount having an upper side thereof coupled to the yoke and having a side surface thereof on which a Z-axis slider is formed;
a X and Z-axis frame provided with a vertical plate on which a Z-axis guide rail is formed, so as to be coupled to the Z-axis slider, and provided with a horizontal plate being coupled at a right angle to a lower side of the vertical plate and having a lower side thereof on which an X-axis slider is formed; and
a main mount having an upper surface thereof on which an X-axis guide rail is formed, so as to be coupled to the X-axis slider.

4. The universal folding test device of claim 3, wherein the rotary plate, the yoke, and the support plate are formed inside an environmental chamber,
the Y-axis rotation actuator is provided with the rotation shaft extending to an outer side of the environmental chamber so that the reduction gear and the rotation motor are formed at the outer side of the environmental chamber, and
the X and Z-axis transfer actuator is provided with a yoke support rod extending to the outer side of the environmental chamber and coupled to a lower side of the yoke so that the yoke mount is formed at the outer side of the environmental chamber, whereby a folding test with the rotary plate and the support plate is able to be performed under environmental conditions.

5. The universal folding test device of claim 4, wherein the yoke mount further comprises a rotation shaft support that extends vertically to the rotation shaft at the outer side of the environmental chamber, so as to support the rotation shaft.

6. The universal folding test device of claim 5, wherein two universal folding test devices for the foldable display are symmetrically formed to face with each other, and
only the rotary plate, the yoke, and the support plate are formed inside the environmental chamber.
